(19) United States
(12) Patent Application Publication
(10) Pub. No.: US 2022/0347011 A1
(43) Pub. Date: Nov. 3, 2022

(54) ICE PACKS, HEATING PADS, COOLERS, AND ICE PACK CARRIERS WITH AN EXTERNAL SURFACE COATED WITH A COATING CONTAINING AN ANTIMICROBIAL AGENT

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 17/853,710

(22) Filed: Jun. 29, 2022

Related U.S. Application Data
(62) Division of application No. 16/269,831, filed on Feb. 7, 2019.
(60) Provisional application No. 62/631,726, filed on Feb. 17, 2018.

(51) Int. Cl. A61F 7/10; C09D 5/14; A01N 55/00; A61F 7/08; B65D 25/14; F25D 3/08; B65D 25/04; A01N 33/12; F25D 3/06

(52) U.S. Cl. CPC A61F 7/10; C09D 5/14; A01N 55/00; A61F 7/08; B65D 25/14; F25D 3/08; B65D 25/04; A01N 33/12; F25D 3/06; A61F 2007/0211

(57) ABSTRACT

An ice pack comprises a plastic sac with an external surface, the plastic sac forming an interior chamber, a substance within the interior chamber capable of absorbing thermal energy, and a coating, which contains one or more antimicrobial agents, on the external surface. The substance can comprise water, including polymer beads mixed with the water. The one or more antimicrobial agents can include a quaternary ammonium ion or salt thereof, such as a silane quaternary ammonium ion or salt thereof. The silane quaternary ammonium ion or salt thereof can be 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, or 3-(trihydroxysilyl)propyldimethyloctadecyl ammonium chloride. Instead of an ice pack, the apparatus comprising the coating containing one or more antimicrobial agents can be an object that is used around injured people and/or perishable items such as heating pads, coolers, and ice pack carriers.

### CROSS-REFERENCE TO RELATED APPLICATIONS

This patent application is a divisional of U.S. patent application Ser. No. 16/269,831, filed on Feb. 7, 2019, entitled ICE PACKS, HEATING PADS, COOLERS, AND ICE PACK CARRIERS WITH AN EXTERNAL SURFACE COATED WITH A COATING CONTAINING AN ANTIMICROBIAL AGENT, which claims priority to and the benefit under 35 U.S.C. § 119(e) of U.S. Provisional Patent Application No. 62/631,726, filed on Feb. 17, 2018, entitled ICE PACKS, HEATING PADS, COOLERS, AND ICE PACK CARRIERS WITH AN EXTERNAL SURFACE COATED WITH A COATING CONTAINING AN ANTIMICROBIAL AGENT,” the entire disclosures of which are incorporated herein by reference.

### BACKGROUND OF THE INVENTION

Ice packs are used for a variety of purposes, including as a cold compress for an injured person and as a supplement or substitute for ice in a cooler to keep perishable items from warming. Similarly, heating pads are used by an injured person to manage pain. Likewise, coolers are typically used, in conjunction with ice packs or ice, to keep perishable items from warming. Ice packs are sometimes shipped and stored in ice pack carriers. Because ice packs, heating pads, coolers, and ice pack carriers are used around injured people and perishable items, these items may act as a vehicle to spread bacteria and other harmful microbes.

### SUMMARY OF THE INVENTION

According to one aspect of the present invention, an ice pack comprises a plastic sac with an external surface, the plastic sac forming an interior chamber, a substance within the interior chamber capable of absorbing thermal energy, and a coating, which contains one or more antimicrobial agents, on the external surface. The substance can comprise water. The substance can further comprise polymer beads mixed with the water. The one or more antimicrobial agents can include a quaternary ammonium ion or salt thereof. The quaternary ammonium ion or salt thereof can be a silane quaternary ammonium ion or salt thereof. The silane quaternary ammonium ion or salt can be 3-(trimethoxysilyl)propyldimethyloctadecyl ammonium ion or 3-(trimethoxysilyl)propyldimethyloctadecyl ammonium chloride, respectively. The silane quaternary ammonium ion or salt can be 3-(trihydroxysilyl)propyldimethyloctadecyl ammonium ion or 3-(trihydroxysilyl)propyldimethyloctadecyl ammonium chloride, respectively.

According to another aspect of the present invention, a heating pad comprises a sac with an external surface, the sac forming an interior chamber, a substance within the interior chamber capable of releasing thermal energy, and a coating, which contains one or more antimicrobial agents, on the external surface. The one or more antimicrobial agents can include a quaternary ammonium ion or salt thereof. The quaternary ammonium ion or salt thereof can be a silane quaternary ammonium ion or salt thereof. The silane quaternary ammonium ion or salt can be 3-(trimethoxysilyl)propyldimethyloctadecyl ammonium ion or 3-(trimethoxysilyl)propyldimethyloctadecyl ammonium chloride, respectively. The silane quaternary ammonium ion or salt can be 3-(trihydroxysilyl)propyldimethyloctadecyl ammonium ion or 3-(trihydroxysilyl)propyldimethyloctadecyl ammonium chloride, respectively.

According to another aspect of the present invention, a cooler comprises a base forming a receptacle and including an outer surface, a lid connected to the base and including an outer surface, and a coating, which contains one or more antimicrobial agents, on the outer surface of the base. The one or more antimicrobial agents can include a quaternary ammonium ion or salt thereof. The quaternary ammonium ion or salt thereof can be a silane quaternary ammonium ion or salt thereof. The silane quaternary ammonium ion or salt can be 3-(trimethoxysilyl)propyldimethyloctadecyl ammonium ion or 3-(trimethoxysilyl)propyldimethyloctadecyl ammonium chloride, respectively. The silane quaternary ammonium ion or salt can be 3-(trihydroxysilyl)propyldimethyloctadecyl ammonium ion or 3-(trihydroxysilyl)propyldimethyloctadecyl ammonium chloride, respectively.

According to another aspect of the present invention, an ice pack carrier comprises an external support forming an interior, a plurality of interior shelves disposed within the interior to support a plurality of ice packs, an external surface, and a coating, which contains one or more antimicrobial agents, on the external surface. The one or more antimicrobial agents can include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion or 3-(trihydroxysilyl)propyldimethyloctadecyl ammonium chloride. The plurality of interior shelves can be supporting a plurality of ice packs, wherein each ice pack of the plurality of ice packs comprises a plastic sac with an interior chamber, an external surface, and a coating, which contains one or more antimicrobial agents, on the external surface, and wherein the one or more antimicrobial agents includes 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion or 3-(trihydroxysilyl)propyldimethyloctadecyl ammonium chloride.

These and other features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is a perspective view of an ice pack including a plastic sac with an external surface and a coating, which contains an antimicrobial agent, on the external surface;

FIG. 2 is a cross-sectional view of the ice pack taken through line II-II of FIG. 1;

FIG. 3 is a magnified view of area III of FIG. 2, illustrating the coating on the external surface;

FIG. 4 is a perspective view of a heating pad including a sac with an external surface and a coating, which contains an antimicrobial agent, on the external surface;

FIG. 5 is a cross-sectional view of the heating pad taken through line V-V of FIG. 4;

FIG. 6 is a magnified view of area VI of FIG. 5, illustrating the coating on the external surface;

FIG. 7 is a perspective view of a cooler including a lid and a base, both having an external surface, and a coating, which contains an antimicrobial agent, on the external surface of the lid and base;

FIG. 8 is a cross-sectional view of the cooler taken through line VIII-VIII of FIG. 7;

FIG. 9 is a magnified view of area IX of FIG. 8, illustrating the coating on the external surface of the base;

FIG. 10 is a perspective view of an ice pack carrier including a plurality of interior shelves and a coating, which contains an antimicrobial agent, on an external surface;

FIG. 11 is a cross-sectional view of the ice pack carrier of FIG. 10 taken along line XI-XI of FIG. 10;

FIG. 12 is a magnified view of area XII of FIG. 11, illustrating the coating on the external surface; and

FIG. 13 is a perspective view of the ice pack carrier including the plurality of interior shelves supporting a plurality of ice packs.

### DETAILED DESCRIPTION

It is to be understood that the disclosure may assume various alternative orientations, except where expressly specified to the contrary. It is also to be understood that the specific devices and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

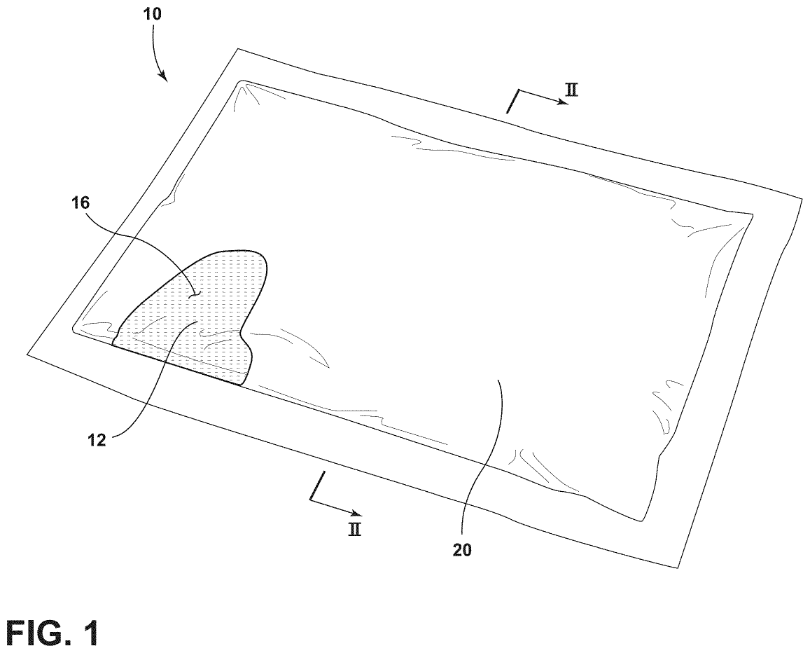

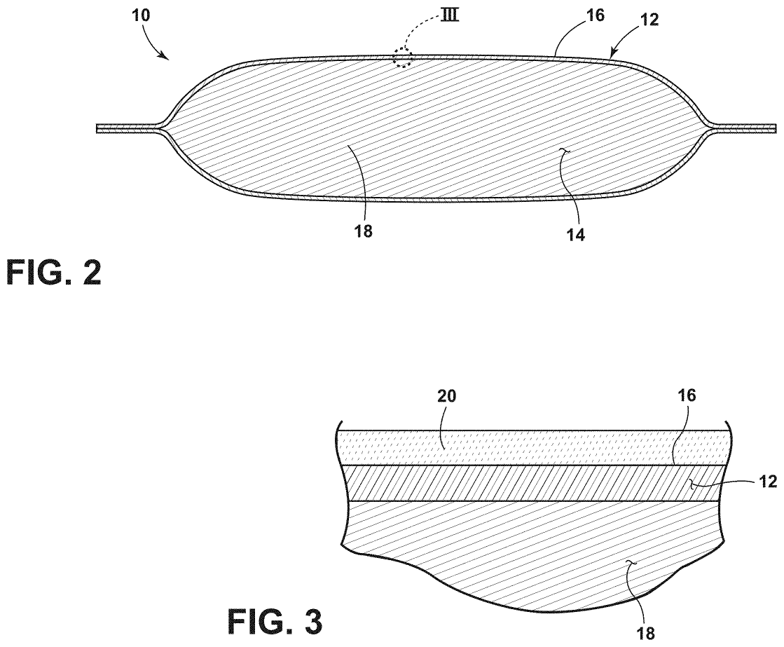

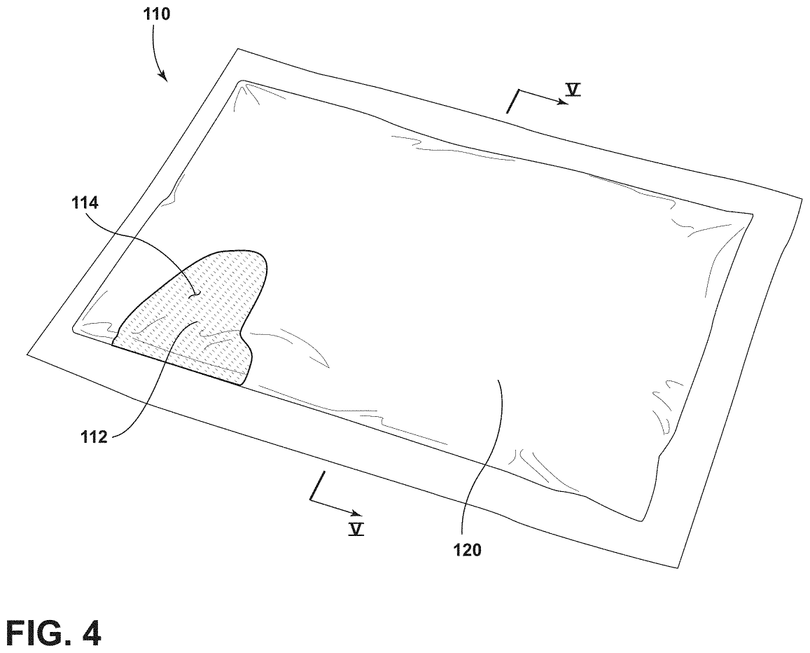

Referring now to FIGS. 1-3, an ice pack 10 includes a plastic sac 12. The plastic sac 12 forms an interior chamber 14. The plastic sac 12 has an external surface 16. The ice pack 10 includes a substance 18 that is capable of absorbing thermal energy. The substance 18 is in the interior chamber 14 of the plastic sac 12. An example of the substance 18 that is capable of absorbing thermal energy is a substance that is a liquid or gel at room temperature but then turns into a solid when frozen. The frozen substance can thus absorb thermal energy from a body that is warmer than the frozen substance. An example of such a liquid is water, which can be frozen into ice, which is then capable of absorbing thermal energy. Polymer beads can be mixed with the liquid in the interior chamber. An example of an ice pack utilizing polymer beads mixed with water is the First Ice® brand of ice packs manufactured by Mertis Products, LLC (San Diego, Calif.).

The ice pack 10 further includes a coating 20, which contains one or more antimicrobial agents, on the external surface 16. An example antimicrobial agent is a quaternary ammonium ion or salt thereof, including a silane quaternary ammonium ion or salt thereof. Among the preferable silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

As an example method of placing the coating 20, which contains one or more antimicrobial agents, on the external surface 16, a solution containing 3-(trihydroxysilyl) propyldimethyloctadecyl ammonium chloride, isopropyl alcohol, and other inactive ingredients can be sprayed onto the external surface 16 of the plastic sac 12 of the ice pack 10. The solution can be sprayed with an electrostatic sprayer. The isopropyl alcohol evaporates, leaving the coating 20 including 3-(trihydroxysilyl) propyldimethyloctadecyl ammonium chloride on the external surface 16. Alternatively, the ice pack 10 can be dipped in a bath of the solution or the solution can otherwise be coated onto the external surface 16 of the plastic sac 12 of the ice pack 10. The solution can contain between 0.01 percent and 10 percent by weight 3-(trihydroxysilyl) propyldimethyloctadecyl ammonium chloride. The solution can contain between 30 percent and 90 percent by weight isopropyl alcohol. More preferably, the solution contains between 0.75 percent and 5 percent by weight 3-(trihydroxysilyl) propyldimethyloctadecyl ammonium chloride, and 60 percent to 70 percent by weight isopropyl alcohol. Most preferably, the solution contains about 2.8 percent by weight 3-(trihydroxysilyl) propyldimethyloctadecyl ammonium chloride and about 60 percent by weight isopropyl alcohol.

In general, the silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution. As for the isopropyl alcohol, the isopropyl alcohol can comprise between 30 percent to 90 percent by weight of the solution. More preferably, the isopropyl alcohol can comprise between 55 percent and 65 percent by weight of the solution. Another example solution comprises (by weight) 60.0 percent isopropyl alcohol, 2.02 percent 3-(trimethoxysilyl)propyldimethyloctadecyl ammonium chloride, and 34.19 percent deionized water.

An alternative solution to place the coating 20, which contains one or more antimicrobial agents, onto the external surface 16, includes 3-(trihydroxysilyl) propyldimethyloctadecyl ammonium chloride and either peracetic acid or hydrogen peroxide or both peracetic acid and hydrogen peroxide. The 3-(trihydroxysilyl) propyldimethyloctadecyl ammonium chloride can be present in an amount between 0.01 percent and 10 percent by weight of the solution. The peracetic acid, if present, can be present in an amount between 0.10 percent and 10 percent by weight of the solution. The hydrogen peroxide, if present, can be present in an amount between 0.10 percent and 10 percent by weight of the solution. For example, the solution can include about 1 percent by weight 3-(trihydroxysilyl) propyldimethyloctadecyl ammonium chloride, about 0.14 percent by weight peracetic acid, and about 0.64 percent by weight hydrogen peroxide. The coating 20 deposits 3-(trihydroxysilyl) propyldimethyloctadecyl ammonium chloride on the external surface 16.

Because the external surface 16 of the ice pack 10 is coated with a coating 20, which contains one or more antimicrobial agents, the ice pack 10 has less ability to act as a vehicle to spread bacteria and other harmful microbes than an ice pack without the coating 20.

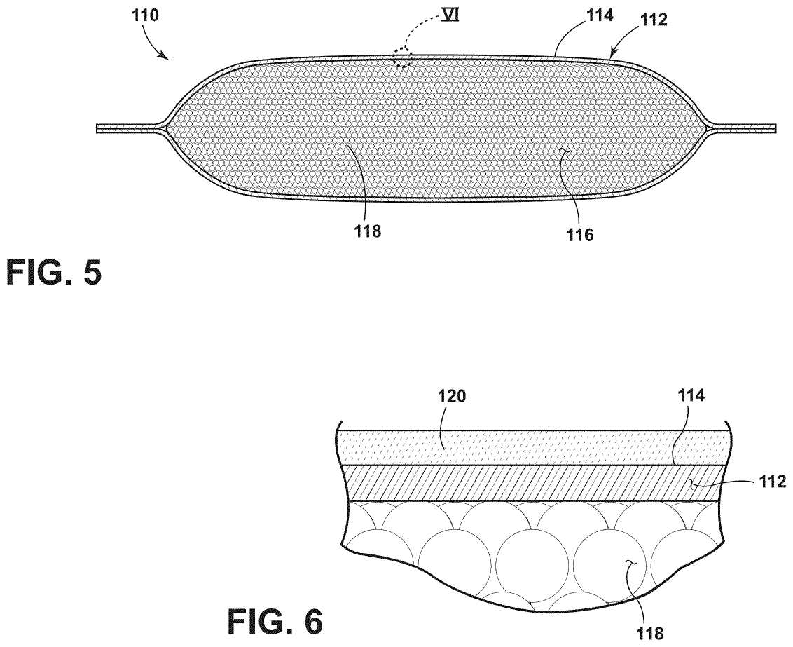

Referring now to FIGS. 4-6, a heating pad 110 has a sac 112 with an external surface 114. The sac 112 forms an interior chamber 116. The heating pad 110 further includes a substance 118 within the interior chamber 116 that is capable of releasing thermal energy. Examples of the substance 118 that is capable of releasing thermal energy include, without limitation, (a) a phase change material, (b) a chemical, which when contacting another chemical, undergoes an exothermic chemical reaction, and (c) a material with a high specific heat capacity, including water, which, when heated, gradually loses thermal energy.

The heating pad 110 further includes a coating 120, which contains one or more antimicrobial agents, on the external surface 114. An example antimicrobial agent is a quaternary ammonium ion or salt thereof, including a silane quaternary ammonium ion or salt thereof. Among the preferable silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

As an example method of placing the coating 120, which contains one or more antimicrobial agents, on the external surface 114, a solution containing 3-(trihydroxysilyl) propyldimethyloctadecyl ammonium chloride, isopropyl alcohol, and other inactive ingredients can be sprayed onto the external surface 114 of the sac 112 of the heating pad 110. The solution can be sprayed with an electrostatic sprayer. The isopropyl alcohol evaporates, leaving the coating 120 including 3-(trihydroxysilyl) propyldimethyloctadecyl ammonium chloride on the external surface 114. Alternatively, the heating pad 110 can be dipped in a bath of the solution or the solution can otherwise be coated onto the external surface 114 of the sac 112 of the heating pad 110. The solution can contain between 0.01 percent and 10 percent by weight 3-(trihydroxysilyl) propyldimethyloctadecyl ammonium chloride. The solution can contain between 30 percent and 90 percent by weight isopropyl alcohol. More preferably, the solution contains between 0.75 percent and 5 percent by weight 3-(trihydroxysilyl) propyldimethyloctadecyl ammonium chloride, and 60 percent to 70 percent by weight isopropyl alcohol. Most preferably, the solution contains about 2.8 percent by weight 3-(trihydroxysilyl) propyldimethyloctadecyl ammonium chloride and about 60 percent by weight isopropyl alcohol.

In general, the silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution. As for the isopropyl alcohol, the isopropyl alcohol can comprise between 30 percent to 90 percent by weight of the solution. More preferably, the isopropyl alcohol can comprise between 55 percent and 65 percent by weight of the solution. Another example solution comprises (by weight) 60.0 percent isopropyl alcohol, 2.02 percent 3-(trimethoxysilyl)propyldimethyloctadecyl ammonium chloride, and 34.19 percent deionized water.

An alternative solution to place the coating 120, which contains one or more antimicrobial agents, onto the external surface 114, includes 3-(trihydroxysilyl) propyldimethyloctadecyl ammonium chloride and either peracetic acid or hydrogen peroxide or both peracetic acid and hydrogen peroxide. The 3-(trihydroxysilyl) propyldimethyloctadecyl ammonium chloride can be present in an amount between 0.01 percent and 10 percent by weight of the solution. The peracetic acid, if present, can be present in an amount between 0.10 percent and 10 percent by weight of the solution. The hydrogen peroxide, if present, can be present in an amount between 0.10 percent and 10 percent by weight of the solution. For example, the solution can include about 1 percent by weight 3-(trihydroxysilyl) propyldimethyloctadecyl ammonium chloride, about 0.14 percent by weight peracetic acid, and about 0.64 percent by weight hydrogen peroxide. The coating 120 deposits 3-(trihydroxysilyl) propyldimethyloctadecyl ammonium chloride on the external surface 114.

Because the external surface 114 of the heating pad 110 is coated with a coating 120, which contains one or more antimicrobial agents, the heating pad 110 has less ability to act as a vehicle to spread bacteria and other harmful microbes than a heating pad without the coating 120.

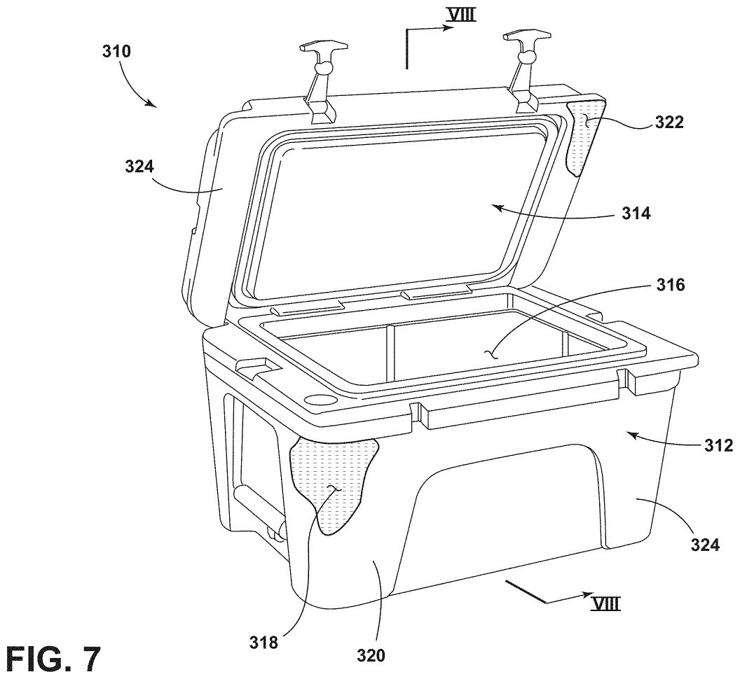

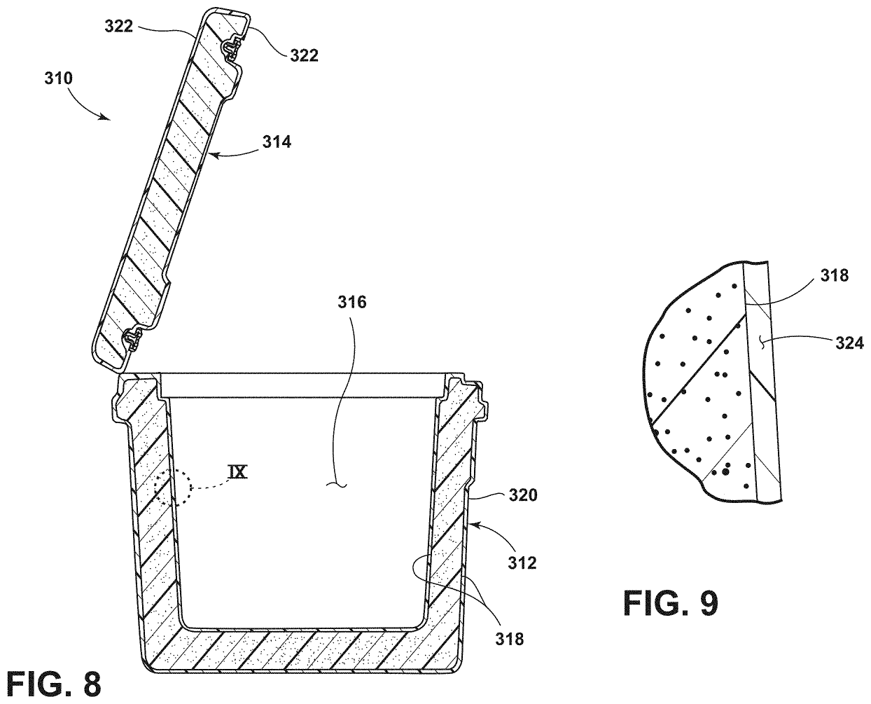

Referring now to FIGS. 7-9, a cooler 310 has a base 312 and a lid 314 connected to the base 312. The base 312 forms a receptacle 316. The base 312 has an outer surface 318, both where the receptacle 316 is formed and on the exterior 320. The lid 314 also has an outer surface 322, both where the lid 314 covers the receptacle 316 and on the exterior. The receptacle 316 can hold an ice pack, including the ice pack 10 according to an embodiment of the present disclosure, ice, and perishable items, such as food. An example of a cooler is the Tundra® 35 distributed by YETI Coolers LLC.

The cooler 310 further includes a coating 324, which contains one or more antimicrobial agents, on the outer surface 318 and outer surface 322. An example antimicrobial agent is a quaternary ammonium ion or salt thereof, including a silane quaternary ammonium ion or salt thereof. Among the preferable silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

As an example method of placing the coating 324, which contains one or more antimicrobial agents, on the outer surface 318 and outer surface 322, a solution containing 3-(trihydroxysilyl) propyldimethyloctadecyl ammonium chloride, isopropyl alcohol, and other inactive ingredients can be sprayed onto the on the outer surface 318 and outer surface 322 of the cooler 310. The solution can be sprayed with an electrostatic sprayer. The isopropyl alcohol evaporates, leaving the coating 324 including 3-(trihydroxysilyl) propyldimethyloctadecyl ammonium chloride on the outer surface 318 and outer surface 322. Alternatively, the cooler 310 can be dipped in a bath of the solution or the solution can otherwise be coated onto the outer surface 318 and outer surface 322. The solution can contain between 0.01 percent and 10 percent by weight 3-(trihydroxysilyl) propyldimethyloctadecyl ammonium chloride. The solution can contain between 30 percent and 90 percent by weight isopropyl alcohol. More preferably, the solution contains between 0.75 percent and 5 percent by weight 3-(trihydroxysilyl) propyldimethyloctadecyl ammonium chloride, and 60 percent to 70 percent by weight isopropyl alcohol. Most preferably, the solution contains about 2.8 percent by weight 3-(trihydroxysilyl) propyldimethyloctadecyl ammonium chloride and about 60 percent by weight isopropyl alcohol.

In general, the silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution. As for the isopropyl alcohol, the isopropyl alcohol can comprise between 30 percent to 90 percent by weight of the solution. More preferably, the isopropyl alcohol can comprise between 55 percent and 65 percent by weight of the solution. Another example solution comprises (by weight) 60.0 percent isopropyl alcohol, 2.02 percent 3-(trimethoxysilyl)propyldimethyloctadecyl ammonium chloride, and 34.19 percent deionized water.

An alternative solution to place the coating 324, which contains one or more antimicrobial agents, on the outer surface 318 and outer surface 322, includes 3-(trihydroxysilyl) propyldimethyloctadecyl ammonium chloride and either peracetic acid or hydrogen peroxide or both peracetic acid and hydrogen peroxide. The 3-(trihydroxysilyl) propyldimethyloctadecyl ammonium chloride can be present in an amount between 0.01 percent and 10 percent by weight of the solution. The peracetic acid, if present, can be present in an amount between 0.10 percent and 10 percent by weight of the solution. The hydrogen peroxide, if present, can be present in an amount between 0.10 percent and 10 percent by weight of the solution. For example, the solution can include about 1 percent by weight 3-(trihydroxysilyl) propyldimethyloctadecyl ammonium chloride, about 0.14 percent by weight peracetic acid, and about 0.64 percent by weight hydrogen peroxide. The coating 324 deposits 3-(trihydroxysilyl) propyldimethyloctadecyl ammonium chloride on the outer surface 318 and outer surface 322.

Because the outer surface 318 and outer surface 322 of the cooler 310 is coated with a coating 324, which contains one or more antimicrobial agents, the cooler 310 has less ability to act as a vehicle to spread bacteria and other harmful microbes than a cooler without the coating 324.

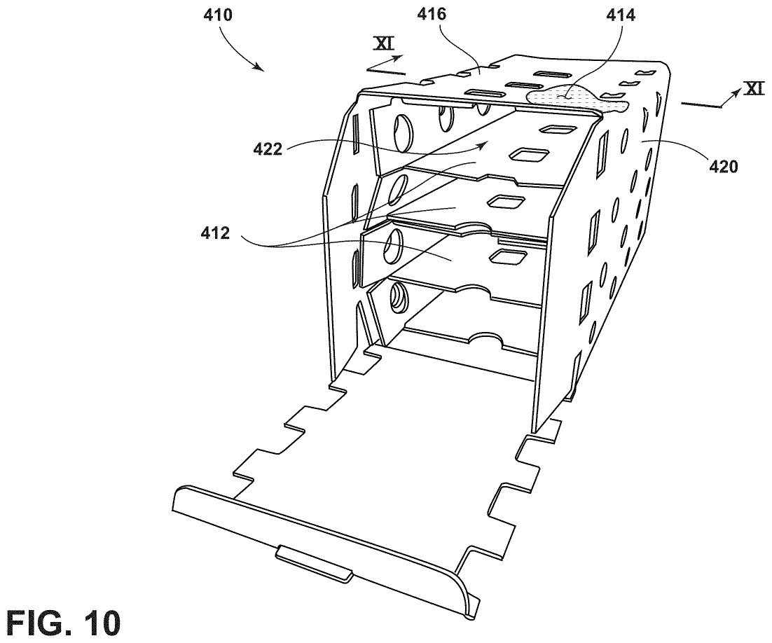

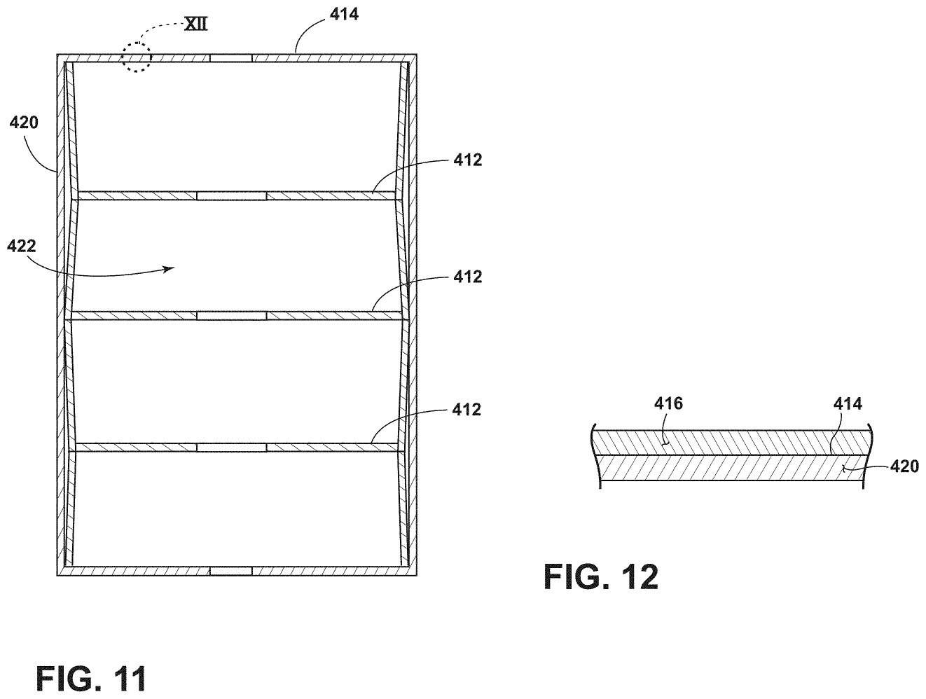

Referring now to FIGS. 10-13, an ice pack carrier 410 includes an external support 420 forming an interior 422, and a plurality of interior shelves 412 disposed within the interior 422. The plurality of interior shelves 412 can support a plurality of ice packs, such as for holding the ice packs flat while freezing the plurality of ice packs or transporting the ice packs. The ice pack carrier further includes an external surface 414.

The ice pack carrier 410 further includes a coating 416, which contains one or more antimicrobial agents, on the external surface 414. An example antimicrobial agent is a quaternary ammonium ion or salt thereof, including a silane quaternary ammonium ion or salt thereof. Among the preferable silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

As an example method of placing the coating 416, which contains one or more antimicrobial agents, on the external surface 414, a solution containing 3-(trihydroxysilyl) propyldimethyloctadecyl ammonium chloride, isopropyl alcohol, and other inactive ingredients can be sprayed onto the external surface 414 of the ice pack carrier 410. The solution can be sprayed with an electrostatic sprayer. The isopropyl alcohol evaporates, leaving the coating 416 including 3-(trihydroxysilyl) propyldimethyloctadecyl ammonium chloride on the external surface 414. Alternatively, the ice pack carrier 410 can be dipped in a bath of the solution or the solution can otherwise be coated onto the external surface 414 of the ice pack carrier 410. The solution can contain between 0.01 percent and 10 percent by weight 3-(trihydroxysilyl) propyldimethyloctadecyl ammonium chloride. The solution can contain between 30 percent and 90 percent by weight isopropyl alcohol. More preferably, the solution contains between 0.75 percent and 5 percent by weight 3-(trihydroxysilyl) propyldimethyloctadecyl ammonium chloride, and 60 percent to 70 percent by weight isopropyl alcohol. Most preferably, the solution contains about 2.8 percent by weight 3-(trihydroxysilyl) propyldimethyloctadecyl ammonium chloride and about 60 percent by weight isopropyl alcohol.

In general, the silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution. As for the isopropyl alcohol, the isopropyl alcohol can comprise between 30 percent to 90 percent by weight of the solution. More preferably, the isopropyl alcohol can comprise between 55 percent and 65 percent by weight of the solution. Another example solution comprises (by weight) 60.0 percent isopropyl alcohol, 2.02 percent 3-(trimethoxysilyl)propyldimethyloctadecyl ammonium chloride, and 34.19 percent deionized water.

An alternative solution to place the coating 416, which contains one or more antimicrobial agents, onto the external surface 414, includes 3-(trihydroxysilyl) propyldimethyloctadecyl ammonium chloride and either peracetic acid or hydrogen peroxide or both peracetic acid and hydrogen peroxide. The 3-(trihydroxysilyl) propyldimethyloctadecyl ammonium chloride can be present in an amount between 0.01 percent and 10 percent by weight of the solution. The peracetic acid, if present, can be present in an amount between 0.10 percent and 10 percent by weight of the solution. The hydrogen peroxide, if present, can be present in an amount between 0.10 percent and 10 percent by weight of the solution. For example, the solution can include about 1 percent by weight 3-(trihydroxysilyl) propyldimethyloctadecyl ammonium chloride, about 0.14 percent by weight peracetic acid, and about 0.64 percent by weight hydrogen peroxide. The coating 416 deposits 3-(trihydroxysilyl) propyldimethyloctadecyl ammonium chloride on the external surface 414.

Because the external surface 414 of the ice pack carrier 410 is coated with a coating 416, which contains one or more antimicrobial agents, the ice pack carrier 410 has less ability to act as a vehicle to spread bacteria and other harmful microbes than an ice pack carrier without the coating 416.

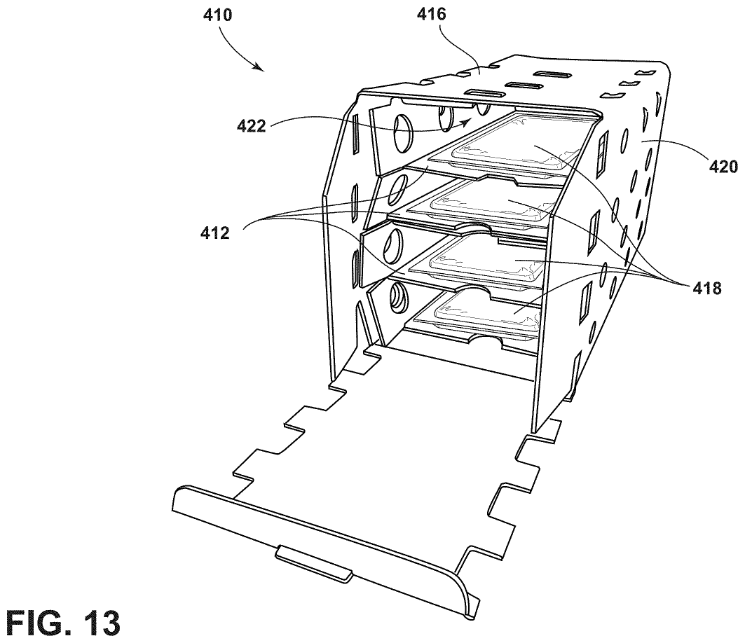

In use, the ice pack carrier 410 can hold a plurality of ice packs (see FIG. 13). For example, the plurality of interior shelves 412 of the ice pack carrier 410 can support a plurality of ice packs 418. Each ice pack of the plurality of ice packs 418 can be the ice pack 10, discussed above.

The one or more antimicrobial agents referred to herein also include biguanide and chemicals that include a biguanide functional group, silver, copper, titanium, chlorhexidine gluconate, and triclosan, and are not limited to quaternary ammonium chemicals such as 3-(trihydroxysilyl) propyldimethyloctadecyl ammonium chloride.

1. A cooler comprising:
a base forming a receptacle and including an outer surface;
a lid connected to the base and including an outer surface; and
a coating, which contains one or more antimicrobial agents, on the outer surface of the base.

2. The cooler of claim 1, wherein the one or more antimicrobial agents includes a quaternary ammonium ion or salt thereof.

3. The cooler of claim 1, wherein the quaternary ammonium ion or salt thereof is a silane quaternary ammonium ion or salt thereof.

4. The cooler of claim 3, wherein the silane quaternary ammonium ion or salt is 3-(trimethoxysilyl)propyldimethyloctadecyl ammonium ion or 3-(trimethoxysilyl)propyldimethyloctadecyl ammonium chloride, respectively.

5. The cooler of claim 3, wherein the silane quaternary ammonium ion or salt is 3-(trihydroxysilyl)propyldimethyloctadecyl ammonium ion or 3-(trihydroxysilyl)propyldimethyloctadecyl ammonium chloride, respectively.

6. The cooler of claim 1, wherein the coating is further disposed on the outer surface of the lid.

7. The cooler of claim 1, wherein the base forms a receptacle, and the coating is further disposed on the outer surface of the receptacle portion of the base.

8. A method comprising:
applying a solution onto an outer surface of a base of a cooler that further comprises a lid connected to the base, the solution comprising a silane quaternary ammonium ion or salt thereof,
wherein, the solution forms a coating comprising the silane quaternary ammonium ion or salt thereof on the outer surface.

9. The method of claim 8 further comprising applying the solution to an outer surface of the lid of the cooler,
wherein, the solution further forms a coating comprising the silane quaternary ammonium ion or salt thereof on the outer surface of the lid.

10. The method of claim 8, wherein the silane quaternary ammonium ion or salt thereof is one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

11. The method of claim 8, wherein
the solution further comprises isopropyl alcohol, and
after the solution is applied, the isopropyl alcohol evaporates.

12. The method of claim 11, wherein
the solution comprises (i) from 0.1 percent to 10 percent by weight of the silane quaternary ammonium ion or salt thereof and (ii) from 30 percent to 90 percent by weight of the isopropyl alcohol.

13. The method of claim 12, wherein the solution comprises from 55 percent to 65 percent by weight of the isopropyl alcohol.

14. The method of claim 12, wherein the solution further comprises deionized water.

15. The method of claim 8, wherein the solution further comprises 0.10 percent to 10 percent by weight of one or more of peracetic acid and hydrogen peroxide.

16. The method of claim 8, wherein the solution is applied via spraying with an electrostatic sprayer.

17. The method of claim 8, wherein the solution is applied via dipping the cooler in a bath of the solution.

18. An ice pack carrier comprising:
an external support forming an interior;
a plurality of interior shelves disposed within the interior configured to support a plurality of ice packs;
an external surface; and
a coating, which contains one or more antimicrobial agents, on the external surface.

19. The ice pack carrier of claim 18, wherein the one or more antimicrobial agents comprises a silane quaternary ammonium ion or salt thereof.

20. The ice pack carrier of claim 19, wherein the silane quaternary ammonium ion or salt thereof comprises one or more of: 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride.

21. The ice pack carrier of claim 19, wherein the silane quaternary ammonium ion or salt thereof comprises one or more of: 3-(trihydroxysilyl)propyldimethyloctadecyl ammonium ion and 3-(trihydroxysilyl)propyldimethyloctadecyl ammonium chloride.
